(19) United States
(12) Patent Application Publication
(10) Pub. No.: US 2023/0213445 A1
(43) Pub. Date: Jul. 6, 2023

(54) OPTICAL WATER QUALITY SENSOR

(71) Applicant: SOLTEAM OPTO, INC., Taoyuan (TW)

(72) Inventors: Chih-Yin HUANG, Taoyuan (TW); Chih-Teng CHENG, Taoyuan (TW); Chin-Feng CHEN, Taoyuan (TW)

(21) Appl. No.: 17/569,721

(22) Filed: Jan. 6, 2022

(51) Int. Cl. G01N 21/47; G01N 33/18

(52) U.S. Cl. CPC G01N 21/47; G01N 33/18; G01N 2201/022

(57) ABSTRACT

An optical water quality sensor includes a holder base having a first light-transmitting portion and a second light-transmitting portion extending in the same direction, a first receiving groove and a second receiving groove respectively extending into the first light-transmitting portion and the second light-transmitting portion and a first light-condensing side and a second light-condensing side respectively located on the inner sides of the first light-transmitting portion and the second light-transmitting portion, and a sensor module with a circuit board assembled in the accommodating space. The circuit board has a first arm plate and a second arm plate respectively inserted into the first receiving groove and the second receiving groove, and a light emitter and a light receiver respectively located on the first arm plate and the second arm plate to face the first light-condensing side and the second light-condensing side.

### BACKGROUND OF THE INVENTION

### 1. Field of the Invention

The present invention relates to water quality sensor technology and more particularly, to an optical water quality sensor, which uses a light emitter and a light receiver to emit and receive light for sensing water quality, and also uses two opposing light-condensing portions to focus light, thereby reducing light scattering and achieving the purpose of improving the accuracy of the judgment of the turbidity of the water.

### 2. Description of the Related Art

With the continuous advancement of the electronic technology era, many convenient home appliances have come out. In daily life, different home appliances can be used for rapid processing to reduce the time-consuming, labor-intensive and imperfect manual operations. In general household appliances that need to use water for operation, such as washing household appliances such as washing machines or dishwashers, they use the water flow inside the machine body with cleaning liquid or detergent to clean the clothes, dishes or tableware and other objects without hands.

Moreover, during the cleaning process of general washing household appliances, the water flow inside the machine body will be affected by the dust, fines, cleaning liquid or detergent and other substances accompanying the objects to be cleaned, which will cause the turbidity of the water to be relatively increased. It is necessary to repeat the cleaning operation many times to achieve the purpose of cleaning the objects to be cleaned. So many more advanced washing appliances will install a water quality sensor. The water quality sensor can transmit or receive the water quality by projecting light to detect the turbidity value of the water quality and determine the turbidity of the water quality. Then, according to the turbidity value, set the current cleaning operation mode of household appliances such as washing machines or dishwashers (for example: increase the number of cleanings or extend the cleaning time, etc.). Its purpose is to improve the washing efficiency and achieve the effect of avoiding water waste. However, when the light emitter in the current water quality sensor projects light outward, it will be refracted, and when the light enters the water flow, it will touch the substance in the water and cause scattering, causing a lot of light energy to lose. Therefore, when the light receiver in the water quality sensor receives light, it cannot accurately know the turbidity value, which affects the accuracy of the judgment. This is the direction that relevant manufacturers in this industry want to study and improve.

Such as the “Liquid Pollution Detection Device” published in the Japanese Patent Publication No. Showa 63-184039 on July 29, Showa 63 (1988), please refer to the side sectional view of the conventional detection device shown in FIG. 6. The beveled edge A1 on one side of the glass rod A is provided with a prism B, and the other side of the prism B is provided with a light-emitting element C. An aluminum tube D is provided on the side relative to the glass rod A, and a light-receiving element D1 is provided at one end of the aluminum tube D. The light-receiving element D1 is electrically connected to the circuit board D2 at the other end of the aluminum tube D. The circuit board D2 is electrically connected with a photodiode D3. The photodiode D3 is located at the reflection position of the beveled edge A1. The glass rod A and the aluminum tube D are respectively covered with a glass tube E1, E2. The above glass rod A, prism B, light-emitting element C, aluminum tube D, light-receiving element D1, circuit board D2, photodiode D3 and glass tubes E1, E2, etc. are assembled inside the housing F. On one side of the housing F is provided with a hood F1 extending to the outside of the glass tubes E1 and E2, so that the glass tubes E1 and E2 are covered inside the hood F1. Use the glass tubes E1 and E2, and the hood F1 to insert into the liquid to be tested (high-temperature edible oil) for testing. Because the glass tubes E1 and E2 are safe in high-temperature liquids and have a heat-insulating effect, they will not adversely affect the prism B, the light-emitting element C, the light-receiving element D1 or the internal circuits, etc. After testing, you only need to wipe and clean the oil stains attached to the outside of the glass tubes E1 and E2, which is convenient and easy to maintain.

The above-mentioned Japanese Patent (Showa 63-184039), its exterior is covered with the hood F1, although it can ensure that the projection line of the light-emitting element C is refracted to the light-receiving element D1 via the glass rod A, however, after the glass rod A receives the bright light of the light-emitting element C, it may scatter the light around and affect the stability of the light-receiving element D1. In addition, the glass rod A and the glass tubes E1 and E2 have high hardness and fragility, and are easily broken and damaged after a little collision, and there are inconvenient defects in use.

### SUMMARY OF THE INVENTION

The present invention has been accomplished under the circumstances in view. It is therefore the main object of the present invention to provide an optical water quality sensor, which comprises a holder base and a sensor module. The holder base comprises a base, an opening located on one side of the base, an accommodating space defined in the base in communication with the opening, a first light-transmitting portion and a second light-transmitting portion extended from an opposite side of the base in the same direction, a first receiving groove and a second receiving groove defined in the base and respectively extended from one side of the accommodating space into the first light-transmitting portion and the second light-transmitting portion, and a first light-condensing side and a second light-condensing side respectively located on opposing inner sides of the first light-transmitting portion and the second light-transmitting portion. The sensor module is assembled in the accommodating space of the base. The sensor module comprises a circuit board positioned in the accommodating space. The circuit board comprises a first arm plate and a second arm plate relatively extending from one side thereof into the first receiving groove and the second receiving groove, and a light emitter and a light receiver respectively provided on the first arm plate and the second arm plate and respectively facing the first light-condensing side and the second light-condensing side. It achieves the purpose of sensing light focusing and projection, better light receiving performance, and improving the accuracy of water turbidity judgment.

It is another object of the present invention to provide an optical water quality sensor, wherein the first light-condensing side and the second light-condensing side are convex and arc-shaped and respectively protruded from respective inner sides of the first light-transmitting portion and the second light-transmitting portion that are extended from the base of the holder base in the same direction, and the holder base further comprises a first light-transmitting surface with a circular arc formed on an outer edge of the first light-condensing side and a second light-transmitting surface with a circular arc formed on an outer edge of the second light-condensing side. Furthermore, the base of the holder base is provided with a first guide path and a second guide path on two opposite sides of the accommodating space. The first guide path and the second guide path respectively extend to the inside of the first receiving groove and the second receiving groove.

It is still another object of the present invention to provide an optical water quality sensor, which further comprises a housing holding the holder base and the sensor module therein. The housing comprises a shell, and an adapter covering one side of the shell. The shell comprises a containment space for accommodating the adapter and the sensor module, two opposite light-transmitting seats protruded from an opposite side thereof far from the adapter, an open channel formed between the two light-transmitting seats, and two accommodating grooves respectively extending into the two light-transmitting seats from two sides of the containment space to accommodate the first light-transmitting portion and the second light-transmitting portion respectively. The adapter comprises a docking slot for one side of the circuit board of the sensor module to be inserted and positioned, and an electrical connector provided on the other side thereof opposite to the docking slot. Furthermore, the base of the holder base is provided with a stop flange on the other side thereof opposite to the first light-transmitting portion and the second light-transmitting portion, and a gasket ring sleeved on the outside of the base to resist at the stop flange. The shell of the housing is provided with a resisting shoulder on the wall surface of the inner containment space opposite to the stop flange of the base for allowing the gasket ring to be located between the stop flange and the resisting shoulder to form a seal.

It is still another object of the present invention to provide an optical water quality sensor, wherein the shell of the housing further comprises a combined space adjacent to the containment space for the adapter to be placed in, and a plurality of buckle grooves provided on the side wall of the combined space. The adapter further comprises a plurality of protruding buckles for engaging the buckle grooves respectively. Furthermore, the resisting shoulder is formed adjacent to the combined space and the containment space. The housing further comprises outer ring groove recessed outside the shell, and a sealing ring sleeved in the outer ring groove to form a sealed state.

### BRIEF DESCRIPTION OF THE DRAWINGS

FIG. 1 is an oblique top elevational view of an optical water quality sensor in accordance with the present invention.

FIG. 2 is an exploded view of the optical water quality sensor in accordance with the present invention.

FIG. 3 is an exploded view in another direction of the optical water quality sensor in accordance with the present invention.

FIG. 4 is a sectional side view of the base of the holder base.

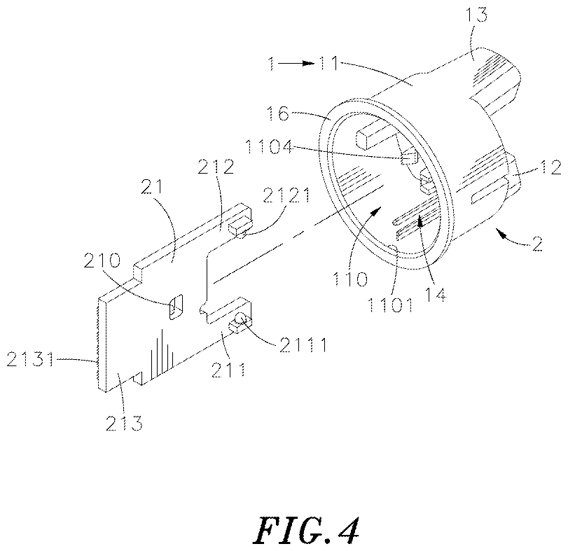

FIG. 5 is a sectional side view of the present invention.

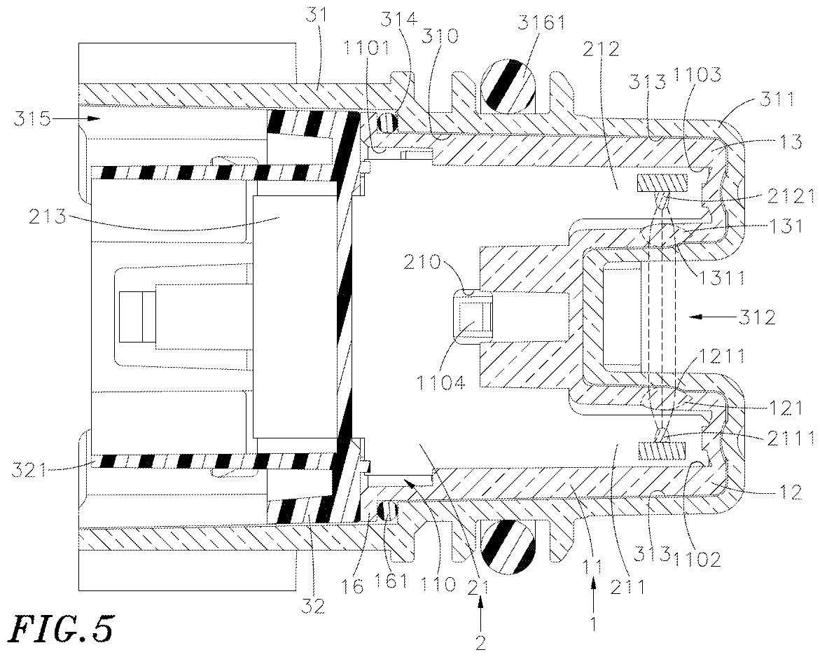

FIG. 6 is a sectional side view of a detection device according to the prior art.

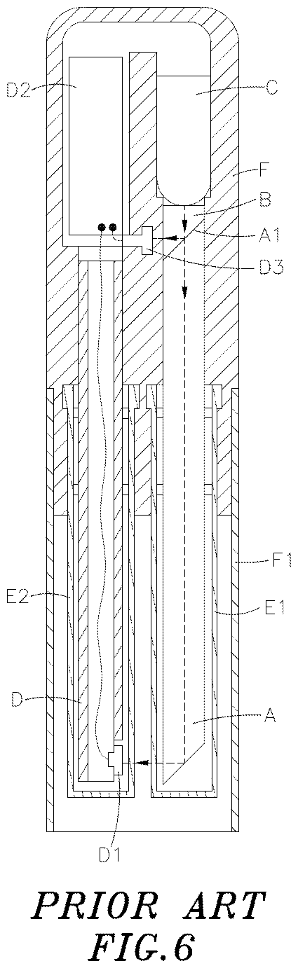

### DETAILED DESCRIPTION OF THE PREFERRED EMBODIMENT

In order to achieve the above-mentioned objects and effects, the technical means and structures adopted by the present invention, the method of implementation, etc., are drawn to illustrate the characteristics and functions of the preferred embodiment of the present invention as follows, for the benefit of a complete understanding.

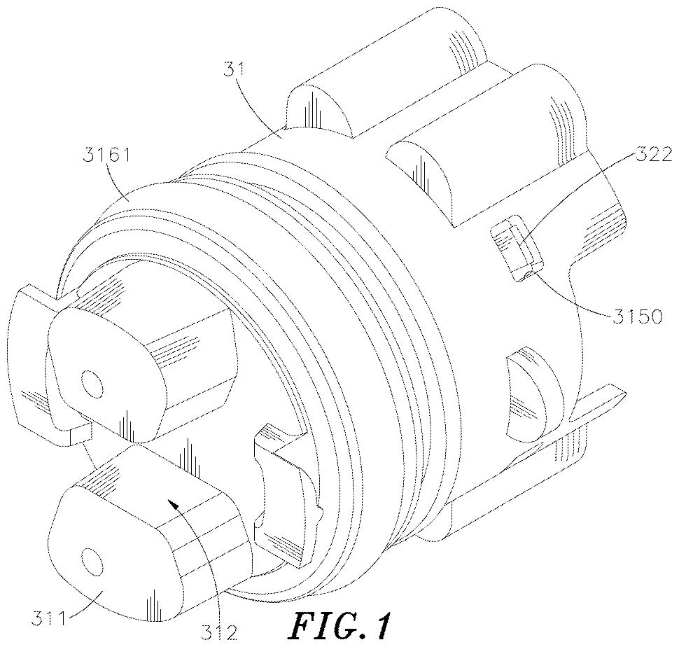

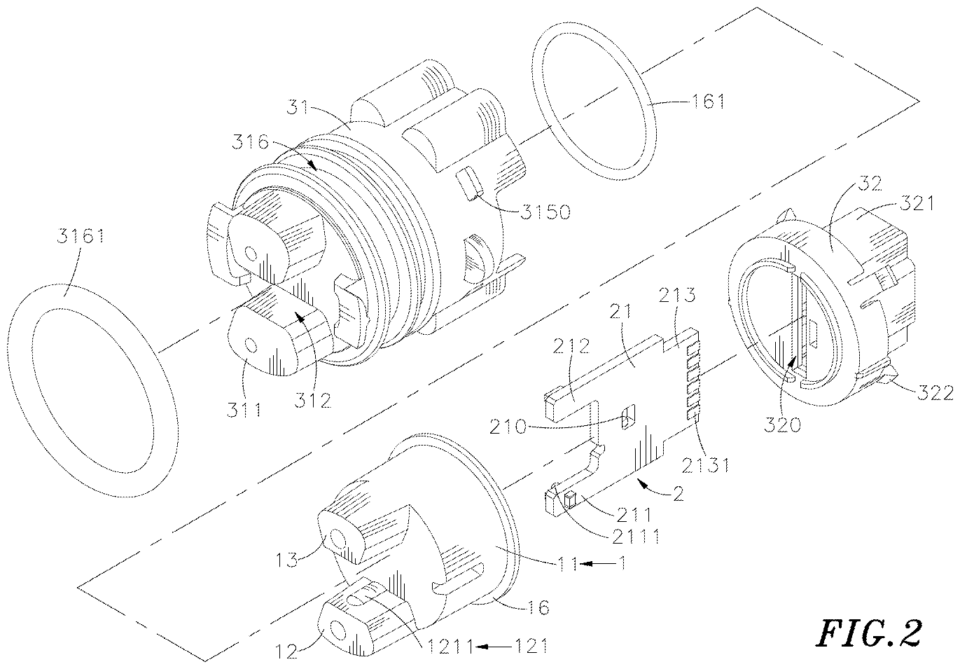

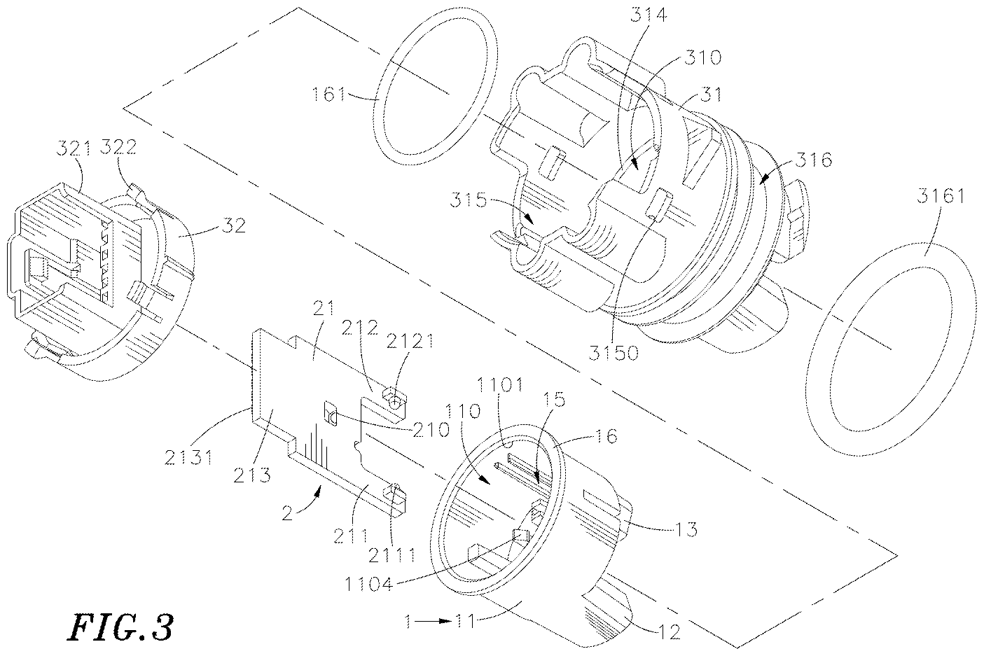

Referring to FIGS. 1-3, which are the oblique top elevational view, the exploded view and the exploded view in another direction of the optical water quality sensor in accordance with the present invention. The optical water quality sensor of the present invention comprises a holder base 1 and a sensor module 2.

The holder base 1 comprises a base 11, an opening 1101 located on one side of the base 11, an accommodating space 110 defined in the base 11 in communication with the opening 1101, a first light-transmitting portion 12 and a second light-transmitting portion 13 extended from an opposite side of the base 11 in the same direction, a first receiving groove 1102 and a second receiving groove 1103 defined in the base 11 and respectively extended from one side of the accommodating space 110 into the first light-transmitting portion 12 and the second light-transmitting portion 13, a limit clasp 1104 located at the adjacent wall surface of the first receiving groove 1102 and the second receiving groove 1103 and protruding in the direction of the opening direction 1101, and a first light-condensing side 121 and a second light-condensing side 131 respectively located on the opposing inner sides of the first light-transmitting portion 12 and the second light-transmitting portion 13.

The sensor module 2 is assembled in the accommodating space 110 of the base 11, comprising a circuit board 21 positioned in the accommodating space 110. The circuit board 21 comprises a first arm plate 211 and a second arm plate 212 relatively extending from one side thereof, a limiting hole 210 located adjacent to the first arm plate 211 and the second arm plate 212, and a light emitter 2111 and a light receiver 2121 respectively provided on the first arm plate 211 and the second arm plate 212 opposite to each other. When placing the sensor module 2 in the accommodating space 110 of the base 11, the first arm plate 211 and the second arm plate 212 can respectively extend into the first receiving groove 1102 and the second receiving groove 1103 to force the limiting hole 210 into engagement with the limit clasp 1104 for the circuit board 21 to be clamped and limited to the accommodating space 110, so that the light emitter 2111 and the light receiver 2121 respectively correspond to the first light-condensing side 121 and the second light-condensing side 131.

The first light-transmitting portion 12 and the second light-transmitting portion 13, which extend in the same direction on one side of the base 11 of the holder base 1, are respectively provided with the convex and arc-shaped first light-condensing sides 121 and second light-condensing side 131 on the opposite inner side. The first light-condensing side 121 and the second light-condensing side 131 have the functions of projecting light and condensing light respectively. On the outer edge of the first light-condensing side 121, a first light-transmitting surface 1211 with a circular arc is formed, and the outer edge of the second light-condensing side 131 is a second light-transmitting surface 1311 with a circular arc. The base 11 of the holder base 1 is provided with a first guide path 14 and a second guide path 15 on two opposite sides of the accommodating space 110. The first guide path 14 and the second guide path 15 respectively extend to the inside of the first receiving groove 1102 and the second receiving groove 1103, so that the two sides of the circuit board 21 of the sensor module 2 can follow the first guide path 14 and the second guide path 15 to slip into the accommodating space 110. The limit clasp 1104 can be engaged in the limiting hole 210 to form a horizontal and axial limit to the circuit board 21, and the first guide path 14 and second guide path 15 are used to limit the two sides of the circuit board 21 in the longitudinal direction, which can prevent the circuit board 21 from being skewed, deviated or rotated after being assembled in the accommodating space 110.

Furthermore, the above-mentioned holder base 1 and sensor module 2 are externally equipped with a housing 3, and the housing 3 comprises a shell 31 and an adapter 32 covering one side of the shell 31. The shell 31 has a containment space 310 for accommodating the adapter 32 and the sensor module 2. On the other side of the shell 31 far from the adapter 32, there are two opposite light-transmitting seats 311 protruding outward, and an open channel 312 is formed between the two light-transmitting seats 311. There are accommodating grooves 313 extending into the two light-transmitting seats 311 from the two sides of the containment space 310 to accommodate the first light-transmitting portion 12 and the second light-transmitting portion 13 respectively. The adapter 32 is provided with a docking slot 320 for a plug portion 213 on the side of the circuit board 21 of the sensor module 2 to be inserted and positioned. The plug portion 213 is provided with a plurality of metal contacts 2131. An electrical connector 321 is provided on the other side of the adapter 32 opposite to the docking slot 320. The electrical connector 321 can be penetrated with a plurality of terminals (not shown), so that one side of the plurality of terminals is extended into the docking slot 320 to contact the plurality of metal contacts 2131 of the plug portion 321. The base 11 of the holder base 1 is provided with a stop flange 16 on the other side opposite to the first light-transmitting portion 12 and the second light-transmitting portion 13. A gasket ring 161 is sleeved on the outside of the base 1 to resist at the stop flange 16. Opposite to the stop flange 16 of the base 11, the wall surface of the inner containment space 310 of the shell 31 of the housing 3 is provided with a resisting shoulder 314 for the gasket ring 161 to be located between the stop flange 16 and the resisting shoulder 314 to form a seal.

The aforementioned housing 3 is provided with a combined space 315 on the side of the adjacent containment space 310 inside the shell 31 for the adapter 32 to be placed in. At least two or more buckle grooves 3150 are provided on the side wall of the combined space 315. At least two or more protruding buckles 322 are provided on the outside of the adapter 32 for engaging the buckle grooves 3150 respectively. The resisting shoulder 314 is formed adjacent to the combined space 315 and the containment space 310. An outer ring groove 316 is recessed outside the shell 31 of the housing 3, and a sealing ring 3161 is sleeved in the outer ring groove 316 to form a sealed state.

The above-mentioned optical water quality sensor of the present invention, in practical application, is implemented by assembling the holder base 1 and the sensor module 2 in the containment space 310 of the shell 31 of the housing 3. Then, assemble the adapter 32 at the combined space 315 on the side of the containment space 310. The plug portion 213 on the side of the circuit board 21 of the sensor module 2 is then inserted into the docking slot 320 of the adapter 32, so that the circuit board 21 and the electrical connector 321 of the adapter 32 are electrically connected. The housing 3 can then be assembled inside the machine body (not shown) of washing household appliances (for example: washing machine, dishwasher, vegetable and fruit washing machine or vegetable washing machine, etc. or other machines and equipment that require water), so that the light-transmitting seats 311 that extend from the shell 31 of housing 3 in the same phase can be placed in the inner working area of the machine body of the washing home appliance. The sealing ring 3161 in the outer ring groove 316 of the shell 31 forms a hermetic contact with the machine body to prevent the internal water flow of the machine body from penetrating into the adapter 32 on the other side of the shell 31. The electrical connector 321 of the adapter 32 on the side of the shell 31 is electrically connected to the operating control system of the machine body (not shown), so that the circuit board 21 can be controlled by the control system for sensing operations. The light emitter 2111 and the light receiver 2121 respectively located on the first arm plate 211 and the second arm plate 212 of the circuit board 21 of the sensor module 2 in the base 11 of the holder base 1 inside the shell 31 can then be operated. At this time, the light emitter 2111 emits the sensing light to the light receiver 2121. After the light receiver 2121 receives the sensing light, it converts the sensing light into a sensing signal and transmits the sensing signal to the circuit board 21, and then the circuit board 21 transmits the sensing signal to the control system of the machine body for washing home appliance through the electrical connector 321, allowing the control system to perform tasks such as sensing, judging, monitoring, and managing the water quality of the washing water.

The light projected from one side of the light emitter 2111 of the above-mentioned sensor module 2 will be magnified by the lens of the first light-transmitting portion 12 and one light-transmitting seat 311, and will then be condensed by the lenses of the second light-transmitting portion 13 and the other light-transmitting seat 311 and then received by the light receiver 2121 to greatly reduce the light projection stroke and the light loss in the path (below 10%). By increasing the light projection intensity of the light emitter 2111 and enhancing the refraction and focusing of the lens of the first light-transmitting portion 12 or the second light-transmitting portion 13, light loss is minimized.

The above-mentioned sensor module 2 is installed in the accommodation space 110 of the base 11 of the holder base 1, and the holder base 1 and the sensor module 2 are installed in the containment space 310 of the shell 31 of the housing 3 to form a double protection for the circuit board 21 and circuit system of the sensor module 2. The first light-transmitting portion 12 and the second light-transmitting portion 13 of the base 11 and the two light-transmitting seats 311 of the shell 31 are in close contact with a very small gap, which can reduce light loss and attenuation of the light between the first light-transmitting portion 12 and the second light-transmitting portion 13 and the two light-transmitting seats 311 during the projection. It can also improve the light projection and receiving effect of the light emitter 2111 and the light receiver 2121. Between the circuit board 21 and the control system of the washing home appliance, it can perform more accurate water quality sensing, judgment and monitoring, and management of washing water.

The electrical connector 321 of the adapter 32 of the housing 3 above is electrically connected to the circuit board 21 of the sensor module 2. The electrical connector 321 can be easily and quickly assembled with the machine body of the washing appliance, which is convenient for the processing process, and at the same time achieves the effects of saving time, labor and reducing operating costs.

During the cleaning operation of the above-mentioned washing household appliance, in addition to the water flow, the operation area contains added cleaning liquid or detergent, and objects to be cleaned (such as: laundry, dishes, tableware or vegetables, fruits, etc.), and impurities on the cleaning fluid or the objects to be cleaned (such as: dust, fines, residues, suspended particles or stains, etc.) will cause the water flow in the working area to be turbid and unclear. When the control system of washing home appliance is operated, the water flow in the working area passes through the open channel 312 formed between the two light-transmitting seats 311 of the shell 31, and drives the light emitter 2111 of the sensor module 2 to project light outwards toward the first light-transmitting portion 12 of the holder base 1. The light emitter 2111 will first project light to the first light-transmitting surface 1211 of the first light-condensing side 121 of the first light-transmitting portion 12, and make the light sequentially pass through the first light-condensing side 121 of the first light-transmitting portion 12. The first light-transmitting surface 1211 is used to vertically refract the light and project it outwards through the light-transmitting seat 311 on one side, so that the light enters the open channel 312. Most of the light will scatter when it comes into contact with impurities in the water stream. The transmitted light that has not been scattered will be projected into the other light-transmitting seat 311. The light enters the second light-condensing side 131 of the base 11 and the second light-transmitting surface 1311 of the second light-condensing side 131 to produce the phenomenon of focusing, thereby reducing the loss of light energy. Then the second light-transmitting surface 1311 is used to project the focused light to the light receiver 2121, so that the light receiver 2121 is used to change the received light into a sensing signal. The sensing signal is then sent to the circuit board 21, and transmitted to the control system of the washing appliance through the electrical connector 321 to determine the turbidity value of the water flow, and then the user can further set or operate the subsequent cleaning operations to enhance the efficiency of washing and have the effect of energy saving and water saving.

The above-mentioned first light-transmitting surface 1211 and second light-transmitting surface 1311 are respectively a design pattern of a circular arc lens. In the first light-transmitting portion 12 and the second light-transmitting portion 13 of the base 11, a spherical design pattern is formed on the inner and outer sides, which can provide the feasibility of production operations, is not prone to deformation, and can reduce the product defect rate. By using automated machines to carry out production, assembly and other consistent operations, it can achieve the purpose of reducing manufacturing time and reducing manufacturing costs.

When the above-mentioned sensor module 2 is used, when the light emitter 2111 at the first arm plate 211 of the circuit board 21 is used to project light outward to the light receiver 2121 at the second arm plate 212, the light will also pass through the first light-transmitting portion 12 and the second light-transmitting portion 13 of the base 11 of the holder base 1, and the two light-transmitting seats 311 of the shell 31. The base 11 and the shell 31 can be made of transparent materials, so they will not affect the stroke and path of the light projection.

The above is only the preferred embodiment of the present invention, and is not limited to the scope of the patent of the present invention. Therefore, all simple modifications and equivalent structural changes made by using the description and schematic content of the present invention should be included within the scope of the patent of the present invention.

To sum up, the optical water quality sensor of the present invention is actually used in practical applications. When it is implemented, it can indeed achieve its efficacy and purpose. Therefore, the present invention is a research and development with excellent practicability, and it meets the requirements of an invention patent application. This patent application is applied in accordance with the law, and hope that the review committee will grant the approval of this application as soon as possible to protect the inventor's hard research and development. If the examiner of the patent office has any doubts, please don't hesitate to write instructions.

What the invention claimed is:

1. An optical water quality sensor, comprising:
a holder base, said holder base comprising a base, an opening located on one side of said base, an accommodating space defined in said base in communication with said opening, a first light-transmitting portion and a second light-transmitting portion extended from an opposite side of said base in the same direction, a first receiving groove and a second receiving groove defined in said base and respectively extended from one side of said accommodating space into said first light-transmitting portion and said second light-transmitting portion, and a first light-condensing side and a second light-condensing side respectively located on opposing inner sides of said first light-transmitting portion and said second light-transmitting portion; and
a sensor module assembled in said accommodating space of said base, said sensor module comprising a circuit board positioned in said accommodating space, said circuit board comprising a first arm plate and a second arm plate relatively extending from one side thereof into said first receiving groove and said second receiving groove, and a light emitter and a light receiver respectively provided on said first arm plate and said second arm plate and respectively corresponding to said first light-condensing side and said second light-condensing side.

2. The optical water quality sensor as claimed in claim 1, wherein said first light-condensing side and said second light-condensing side are convex and arc-shaped and respectively protruded from respective inner sides of said first light-transmitting portion and said second light-transmitting portion that are extended from said base of said holder base in the same direction, and said holder base further comprises a first light-transmitting surface with a circular arc formed on an outer edge of said first light-condensing side and a second light-transmitting surface with a circular arc formed on an outer edge of said second light-condensing side.

3. The optical water quality sensor as claimed in claim 1, wherein said base of said holder base is provided with a first guide path and a second guide path on two opposite sides of said accommodating space, said first guide path and said second guide path respectively extending to the inside of said first receiving groove and said second receiving groove.

4. The optical water quality sensor as claimed in claim 1, further comprising a housing holding said holder base and said sensor module therein, said housing comprising a shell and an adapter covering one side of said shell, said shell comprising a containment space for accommodating said adapter and said sensor module, two opposite light-transmitting seats protruded from an opposite side thereof far from said adapter, an open channel formed between said two light-transmitting seats and two accommodating grooves respectively extending into said two light-transmitting seats from two sides of said containment space to accommodate said first light-transmitting portion and said second light-transmitting portion respectively, said adapter comprising a docking slot for one side of said circuit board of said sensor module to be inserted and positioned and an electrical connector provided on the other side thereof opposite to said docking slot.

5. The optical water quality sensor as claimed in claim 4, wherein a base of said holder base is provided with a stop flange on the other side thereof opposite to said first light-transmitting portion and said second light-transmitting portion, and a gasket ring sleeved on the outside of said base to resist at said stop flange; said shell of said housing is provided with a resisting shoulder on a wall surface of said inner containment space opposite to said stop flange of said base for allowing said gasket ring to be located between said stop flange and said resisting shoulder to form a seal.

6. The optical water quality sensor as claimed in claim 4, wherein said shell of said housing further comprises a combined space adjacent to said containment space for said adapter to be placed in, and a plurality of buckle grooves provided on a side wall of said combined space; said adapter further comprises a plurality of protruding buckles for engaging said buckle grooves respectively: a resisting shoulder is formed adjacent to said combined space and said containment space.

7. The optical water quality sensor as claimed in claim 4, wherein said circuit board comprises a plug portion extended from one side thereof and plugged into said docking slot of said adapter to electrically connect with said electrical connector.

8. The optical water quality sensor as claimed in claim 1, wherein a housing further comprises outer ring groove recessed outside a shell, and a sealing ring sleeved in said outer ring groove to form a sealed state.
